SEQUENCE LISTING (21 sequences)

SEQ ID NO: 1 - protein, 16 aa, Homo sapiens
FSGSNSGNTATLTISR

SEQ ID NO: 2 - protein, 7 aa, Homo sapiens
PSGIPER

SEQ ID NO: 3 - protein, 10 aa, Homo sapiens
AGVETTTPSK

SEQ ID NO: 4 - protein, 20 aa, Homo sapiens
ATLVCLISDFYPGAVTYAWK

SEQ ID NO: 5 - protein, 15 aa, Homo sapiens
SYSCQVTHEGSTVEK

SEQ ID NO: 6 - protein, 15 aa, Homo sapiens
YAASSYLSLTPEQWK

SEQ ID NO: 7 - protein, 94 aa, Homo sapiens
SYVVTQPPSVSVAPGQTAKITCGGYNIGSKNMHWYQQTPGQAPVLVVYDDAERPSGIPERFSGSNSGNTATLTISRVEAGDEADYYCQVWDTTY

SEQ ID NO: 8 - protein, 12 aa, Homo sapiens
VVFGGGTKLTVL

SEQ ID NO: 9 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 15 aa, Homo sapiens
ACEVTHQGLSSPVTK

SEQ ID NO: 11 - protein, 14 aa, Homo sapiens
CEVTHQGLSSPVTK

SEQ ID NO: 12 - protein, 14 aa, Homo sapiens
DSTYSLSSTLTLSK

SEQ ID NO: 13 - protein, 19 aa, Homo sapiens
HKVYACEVTHQGLSSPVTK

SEQ ID NO: 14 - protein, 7 aa, Homo sapiens
SFNRGEC

SEQ ID NO: 15 - protein, 16 aa, Homo sapiens
SGTASVVCLLNNFYPR

SEQ ID NO: 16 - protein, 18 aa, Homo sapiens
TVAAPSVFIFPPSDEQLK

SEQ ID NO: 17 - protein, 18 aa, Homo sapiens
VDNALQSGNSQESVTEQD

SEQ ID NO: 18 - protein, 20 aa, Homo sapiens
VDNALQSGNSQESVTEQDSK

SEQ ID NO: 19 - protein, 34 aa, Homo sapiens
VDNALQSGNSQESVTEQDSKDSTYSLSSTLTLSK

SEQ ID NO: 20 - protein, 17 aa, Homo sapiens
VYACEVTHQGLSSPVTK

SEQ ID NO: 21 - protein, 16 aa, Homo sapiens
YACEVTHQGLSSPVTK

(19) United States
(12) Patent Application Publication
(10) Pub. No.: US 2012/0135542 A1
(43) Pub. Date: May 31, 2012

(54) METHODS AND MATERIALS FOR DIAGNOSING LIGHT CHAIN AMYLOIDOSIS

(75) Inventors: Marina Ramirez-Alvarado, Rochester, MN (US); Christopher J. Ward, Rochester, MN (US); Nelson Leung, Rochester, MN (US)

(73) Assignee: MAYO FOUNDATION FOR MEDICAL EDUCATION AND RESEARCH, Rochester, MN (US)

(21) Appl. No.: 13/376,328

(22) PCT Filed: Jun. 3, 2010
(86) PCT No.: PCT/US10/37253

§ 371 Date: Feb. 7, 2012

Related U.S. Application Data
(60) Provisional application No. 61/184,702, filed on Jun. 5, 2009.

(51) Int. Cl. G01N 33/563; G01N 33/53

(52) U.S. Cl. 436/501

(57) ABSTRACT

This document relates to methods and materials involved in diagnosing light chain amyloidosis. For example, methods and materials for using exosomes to diagnose a mammal (e.g., a human) as having immunoglobulin light chain amyloidosis are provided.

### CROSS-REFERENCE TO RELATED APPLICATIONS

This application claims priority to U.S. Provisional Application Ser. No. 61/184,702, filed on Jun. 5, 2009.

### BACKGROUND

1. Technical Field

This document relates to methods and materials involved in diagnosing light chain amyloidosis. For example, this document relates to methods and materials for using exosomes to diagnose a mammal (e.g., a human) as having immunoglobulin light chain amyloidosis.

2. Background Information

Exosomes are small extracellular vesicles (about 40-100 nm in diameter) that originate from renal epithelial cells including glomerular podocytes, renal tubule cells, and the cells lining the urinary draining system (Pisitkun et al., Proc. Natl. Acad. Sci. USA, 101:13368-73 (2004)). Exosomes are formed as part of the multivesicular body (MVB) pathway in which intraluminal vesicles (ILVs) progressively accumulate during endosome maturation. They are formed by inward budding and scission of vesicles from the limiting endosomal membranes (Vella et al., Eur. Biophys. J., 37:323-32 (2008)). Exosomes are released from the MVB lumen into the extracellular environment during exocytosis. During this process, certain cytosolic proteins are incorporated into the invaginating membrane, engulfed in these vesicles, thereby maintaining the same topological orientation as the plasma membrane.

Exosome functionality seems to be determined by cell-type specific polypeptides. The presence of exosomes in serum and other body fluids such as malignant effusions, urine, and bronchoalveolar lavage suggests their involvement in physiological and pathological processes. (Simpson et al., Proteomics, 8:4083-99 (2008)). Their ability to bind target cells indicates that they may be capable of modulating selected cellular activities. Exosomes are thought to be involved with the removal of unwanted proteins and transfer of pathogens (i.e., HIV) between cells.

### SUMMARY

This document relates to methods and materials involved in diagnosing immunoglobulin light chain amyloidosis. For example, this document provides methods and materials for using exosomes to diagnose a mammal (e.g., a human) as having active immunoglobulin light chain amyloidosis. Diagnosing a mammal as having active immunoglobulin light chain amyloidosis using the methods and materials provided herein can allow physicians to treat mammals properly during active periods of the condition. In addition, the methods and materials provided herein can be used to follow the progression of immunoglobulin light chain amyloidosis.

In general, one aspect of this document features a method for assessing a mammal for active immunoglobulin light chain amyloidosis. The method comprises, or consists essentially of, determining whether or not a urine sample from the mammal contains exosomes having immunoglobulin light chain polypeptides associated as complexes with a molecular weight greater than 150 kDa, wherein the presence of the exosomes indicates that the mammal has active immunoglobulin light chain amyloidosis, and wherein the absence of the exosomes indicates that the mammal does not have active immunoglobulin light chain amyloidosis. The mammal can be a human. The molecular weight can be greater than 200 kDa. The exosomes can contain a podocin polypeptide. The method can comprise diagnosing the mammal as having active immunoglobulin light chain amyloidosis if the exosomes are present and diagnosing the mammal as not having active immunoglobulin light chain amyloidosis if the exosomes are absent. The determining step can comprise using an anti-immunoglobulin light chain antibody to detect the presence or absence of the exosomes. The determining step can comprise performing a Western blot.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a photograph of a Western blot showing free light chain immunoreactive polypeptides for active AL patient #1 (a), active AL patient #2 (b), active AL patient #3 (c), active AL patient #4 (d), and active AL patient #5 (e). 15% SDS-PAGE gels under reducing conditions were used.

FIG. 2 is a photograph of a Western blot showing free light chain immunoreactive polypeptides for MM patient #1 (a), MM patient #2 (b), MM patient #3 (c), MM patient #4 (d), and MM patient #5 (e). 15% SDS-PAGE gels under reducing conditions were used.

FIG. 3 is a photograph of a Western blot showing free light chain immunoreactive polypeptides for a control patient with proteinuria (a), a control patient with MOUS lambda (b), and a control patient with IgA nephropathy lambda (c). 15% SDS-PAGE gels under reducing conditions were used.

FIG. 4 is a photograph of a Western blot showing free light chain immunoreactive polypeptides for MIDD patient #1 (c) and MIDD patient #2 (d). 15% SDS-PAGE gels under reducing conditions were used.

FIG. 5 is a photograph of a proteomic analysis of light chains in selected samples using 1D-gel analysis (active AL patient #1, fraction 5 (a); normal control (b); and MIDD patient #1 (c)). 4-15% gradient SDS-PAGE were used. Polypeptides were stained using silver stain. Slices were cut, and trypsin digestion products were analyzed by mass spectrometry. The sequences found in the slices are shown using the one-letter amino acid code, and the different polypeptides found in A are shaded with different shades.

FIG. 6 is a collection of representative immuno-gold labeling images of exosomes from a normal control, fraction 8.

FIG. 7 is a collection of representative immuno-gold labeling images of exosomes from active AL patient #2, fractions 5 and 9.

FIG. 8 is a collection of representative immuno-gold labeling images of exosomes from MIDD patient #2, fractions 5 and 8.

FIG. 9 is a collection of representative immuno-gold labeling images of exosomes from an MM patient, fractions 2, 5, and 9.

FIG. 10 is a photograph of a Western blot corresponding to membrane bound light chain and cytoplasmic light chains from exosomes of representative samples. The fractions used for this experiment were the following: Fraction 8 for normal control, fraction 5 for AL patient #1 and AL patient #2, fraction 8 for LCDD patient #1, fraction 5 for LCDD patient #2. No immuno-reactivity was found for MM fraction 2 at the time of this experiment.

FIG. 11 is a photograph of a Western blot of AL patient #1 exosomes using an anti-podocin antibody.

FIG. 12 is a graph plotting the percent of light chains in the indicated samples.

### DETAILED DESCRIPTION

This document provides methods and materials related to diagnosing mammals as having immunoglobulin light chain amyloidosis. For example, this document provides methods and materials involved in using exosomes (e.g., urinary exosomes) as a marker to determine whether or not a mammal (e.g., a human) has active immunoglobulin light chain amyloidosis. As described herein, mammals having active immunoglobulin light chain amyloidosis can produce and secrete exosomes (e.g., urinary exosomes) having immunoglobulin light chain polypeptides that form complexes having a molecular weight greater than 150 kDa (e.g., greater than 160 kDa, greater than 170 kDa, greater than 180 kDa, greater than 190 kDa, greater than 200 kDa, greater than 210 kDa, greater than 220 kDa, greater than 230 kDa, greater than 240 kDa, greater than 250 kDa, greater than 260 kDa, greater than 270 kDa, greater than 280 kDa, or more). Normal mammals without immunoglobulin light chain amyloidosis as well as mammals with inactive immunoglobulin light chain amyloidosis can lack such immunoglobulin light chains in their urinary exosomes.

The terms “urinary exosome” and “urinary exosome-like vesicle” can be used interchangeably herein and refer to small extracellular vesicles (about 40-100 nm in diameter) that originate from renal epithelial cells. Immunoglobulin light chain polypeptides can be lambda or kappa light chain polypeptides.

Any appropriate method can be used to obtain exosomes (e.g., urinary exosomes). For example, the methods and materials described herein as well as those described elsewhere (see, e.g., Hogan et al., J. Am. Soc. Nephrol., 20(2):278-88 (2009) and Sikkink and Ramirez-Alvarado, Amyloid, 15:29-39 (2008)) can be used to obtain urinary exosomes from a urine sample. In some cases, lectins can be used as described in U.S. Provisional Patent Application No. 61/184,663 (Attorney Docket No.: 07039-0938P01), filed on the same day as the instant document, can be used to obtain exosomes (e.g., urinary exosomes) from a sample. For example, a potato (Solanum tuberosum) lectin or a Maackia amurensis II lectin can be used to obtain polycystic kidney disease exosome-like vesicles (PKD-ELV5). PKD-ELVs are exosome-like vesicles that contain one or more polypeptides (e.g., polycystin-1 (PC1), polycystin-2 (PC2), or fibrocystin/polyductin (FCP)) from the polycystic kidney disease gene. Any appropriate volume of urine can be used to obtain exosomes (e.g., urinary exosomes). For example, 50, 75, 100, 125, 150, 175, 200, 225, 250, or more mL of urine can be used.

In some cases, a urine sample can be processed before exosomes are isolated. For example, a urine sample can be subjected to a centrifugation step to remove cells or debris. Such a centrifugation step can include spinning the sample (e.g., a urine sample) at between 12,000 to 22,000 g (e.g., about 17,000 g) for between 10 and 20 minutes (e.g., about 15 minutes). In some cases, a urine sample or the resulting supernatant from a centrifugation step can be subjected to the exosome isolation process or can be subjected to a dialysis step. Such a dialysis step can include dialyzing a urine sample or the supernatant resulting from centrifugation of a urine sample against a large volume (e.g., a volume between 3 to 5 L such as about 4 L) of buffer (e.g., 100 mM MES buffer, pH 6.0) using a membrane having a molecular weight cutoff between about 5,000 Da and about 125,000 Da (e.g., between about 5,000 Da and about 100,000 Da, between about 5,000 Da and about 75,000 Da, between about 5,000 Da and about 50,000 Da, between about 10,000 Da and about 125,000 Da, between about 15,000 Da and about 125,000 Da, between about 25,000 Da and about 125,000 Da, or between about 50,000 Da and 100,000 Da). For example, a membrane have a molecular weight cutoff about 10,000 Da or about 100,000 Da can be used. This dialysis step can be repeated one, two, three, four, or more times. Once obtained, a urine sample (e.g., a centrifuged, dialyzed urine sample, a centrifuged, undialyzed urine sample, or an unprocessed urine sample) can be subjected to an exosome isolation step as described herein (e.g., centrifugation or chromatography).

Once exosomes (e.g., urinary exosomes) are obtained, any appropriate method can be use to determine whether or not they contain immunoglobulin light chain polypeptides that form complexes having a molecular weight greater than 150 kDa. For example, Western blot analysis or proteomics can be used to determine whether or not exosomes contain immunoglobulin light chain polypeptides that form complexes having a molecular weight greater than 150 kDa. The presence of exosomes (e.g., urinary exosomes) having immunoglobulin light chain polypeptides that form complexes having a molecular weight greater than 150 kDa (e.g., greater than 175, 200, 225, 250, 275, or more kDa) can indicate that the mammal has active immunoglobulin light chain amyloidosis. The absence of exosomes (e.g., urinary exosomes) having immunoglobulin light chain polypeptides that form complexes having a molecular weight greater than 150 kDa (e.g., greater than 175, 200, 225, 250, 275, or more kDa) can indicate that the mammal does not have active immunoglobulin light chain amyloidosis.

The methods and materials provided herein can be used to diagnose any type of mammal as having or lacking active immunoglobulin light chain amyloidosis including, without limitation, dogs, cats, horses, cows, goats, monkeys, and humans.

This document also provides methods and materials to assist medical or research professionals in determining whether or not a mammal has active immunoglobulin light chain amyloidosis. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether or not a mammal (e.g., a human) contains exosomes (e.g., urinary exosomes) having immunoglobulin light chain polypeptides that form complexes having a molecular weight greater than 150 kDa, and (2) communicating information about the presence or absence of such exosomes to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### Use of Light Chain Containing Exosomes to Diagnose Immunoglobulin Light Chain Amyloidosis

Urine samples from patients with plasma cell dyscrasias were obtained from a urine bank that gathers urine samples from patients who gave research use consent for specimens that would otherwise be considered waste.

Exosomes were extracted and fractionated from urine collected and processed as described elsewhere (Hogan et al., J. Am. Soc. Nephrol., 20(2):278-88 (2009) and Sikkink and Ramirez-Alvarado, Amyloid, 15:29-39 (2008)). Briefly, 200 mL of urine were dialyzed against distilled deionized water using a dialysis membrane with 10,000 Da Molecular weight cut off. The urine was then filtered using 0.2 μm membrane, and a Complete® EDTA-free protease inhibitor tablet was added. 0.02% NaN₃ was added, and the sample was stored at 4° C. until it was processed the next day. The sample was then centrifuged at 45,000 rpm in a T-647.5 rotor for 2 hours at 4° C. The glassy pellet was then resuspended in 0.25 mL of 0.25 M sucrose in 20 mM HEPES, pH 7.5 with Complete® EDTA-free protease inhibitor. The suspension was sonicated in a cup-horned sonicator (550 sonic dismembranator) for 15 seconds. Two 5-30% sucrose Deuterium oxide gradients were prepared and overlayed with 0.125 mL of exosome preparation and then centrifuged at 40,000 rpm for 24 hours. 6 mm fractions were removed from the gradient using a Biocomp® Gradient Station (Biocomp, Canada).

The fractions were blotted using the sheep free kappa or lambda light chain antibodies from the Binding Site (Birmingham, United Kingdom; (1:2000)). For polycystin 1, a mouse monoclonal antibody (7e12; anti-LRR PC1) was used (1:500). For the identification of glomerular exosomes, a rabbit anti-podocin antibody from Sigma was used (1:2000). Immuno gold electron microscopy was performed as described elsewhere (van Niel et al., Gastroenterology, 121:337-49 (2001)). Exosome specimens were loaded on 300 mesh copper formvar/carbon grids and incubated with sheep anti human kappa free light chain antibody from the Binding Site (Birmingham, United Kingdom; (1/10 and 1/20 dilution)) in phosphate buffered saline at 4° C. overnight. The grids were then incubated with a 10 nm anti-goat immunoglobulin G gold secondary antibody (1/30 dilution) for 2 hours at room temperature. After washing, the specimens were further fixed in 1% glutaraldehyde/PBS, and stained and embedded with 2% methylcellulose solution containing 0.4% uranyl acetate for 5 minutes. Specimen were then air dried and were imaged with a JEOL 1400 transmission electron microscope operating at 80 kV.

The SDS-PAGE gel bands were prepared for mass spectrometry analysis using the following procedures. Silver stained gel bands were destained with 15 mM potassium ferricyanide and 50 mM sodium thioisulfate in water until clear, then rinsed with water several times to remove all color as described elsewhere (Gharandaghi et al., Electrophoresis, 20:601-5 (1999)). The bands were reduced with 30 mM DTT/50 mM Tris, pH 8.1 at 55° C. for 40 minutes and alkylated with 40 mM iodoacetamide at room temperature for 40 minutes in the dark. Polypeptides were digested in-situ with 30 μL, (0.004 μg/L) trypsin (Promega Corporation, Madison Wis.) in 20 mM Tris pH 8.1/0.0002% Zwittergent 3-16, at 37° C. overnight, followed by polypeptide extraction with 40 μL of 2% trifluoroacetic acid, then 60 μL of acetonitrile. The pooled extracts were concentrated to less than 5 μL on a SpeedVac spinning concentrator (Savant Instruments, Holbrook N.Y.) and then brought up in 0.15% formic acid/0.05% trifluoroacetic acid for polypeptide identification by nano-flow liquid chromatography electrospray tandem mass spectrometry (nanoLC-ESI-MS/MS) using a ThermoFinnigan LTQ Orbitrap Hybrid Mass Spectrometer (ThermoElectron Bremen, Germany) coupled to an Eksigent nanoLC-2D HPLC system (Eksigent, Dublin, Calif.). The digested polypeptide mixture was loaded onto a 250 mL OPTI-PAK trap (Optimize Technologies, Oregon City, Oreg.) custom packed with Michrom Magic C8 solid phase (Michrom Bioresources, Auburn, Calif.). Chromatography was performed using 0.2% formic acid in both the A solvent (98% water/2% acetonitrile) and B solvent (80% acetonitrile/10% isopropanol/10% water), and running a 5% B to 45% B gradient over 60 minutes at 350 mL/minute through a Michrom Magic C18 (75 μm×150 mm) packed tip capillary column. The LTQ Orbitrap mass spectrometer experiment was set to perform a FT full scan from 375-1600 m/z with resolution set at 60,000 (at 400 m/z), followed by linear ion trap MS/MS scans on the top four [M+2H]²⁺ or [M+3H]³⁺ ions. Dynamic exclusion was set to two repeats of the same ion which was then placed on an exclusion list for 20 seconds. The lock-mass option was enabled for the FT full scans using the ambient air polydimethylcyclosiloxane (PCM) ion of m/z=355.069933 for real time internal calibration giving <2 ppm mass tolerances of the precursor masses Olsen et al., Mol. Cell. Proteomics, 4:2010-21 (2005)).

Tandem mass spectra were extracted and charge state deconvoluted by BioWorks version 3.2. Deisotoping was not performed. All MS/MS samples were analyzed using Mascot (Matrix Science, London, UK; version 2.2.04), Sequest (ThermoFinnigan, San Jose, Calif.; version 27, rev. 12) and X! Tandem (world wide web at “thegpm.org”; version 2006.09.15.3). X! Tandem was set up to search the SprotProt plus reverse database (699052 entries) assuming the digestion enzyme semiTrypsin. Sequest was set up to search the SprotProt plus reverse database (699052 entries) also assuming trypsin. Mascot was set up to search the SprotProt plus reverse database (699052 entries) assuming the digestion enzyme trypsin. Mascot and X! Tandem were searched with a fragment ion mass tolerance of 0.80 Da and a parent ion tolerance of 10.0 PPM. Sequest was searched with a fragment ion mass tolerance of 0.80 Da and a parent ion tolerance of 0.011 Da. Oxidation of methionine and iodoacetamide derivative of cysteine were specified in Mascot, Sequest, and X! Tandem as variable modifications.

Scaffold (version Scaffold-2_{—}00_{—}02, Proteome Software Inc., Portland, Oreg.) was used to validate MS/MS based polypeptide identifications. Peptide identifications were accepted if they can be established at greater than 95.0% probability as specified by the Peptide Prophet algorithm (Keller et al., Anal. Chem., 74:5383-92 (2002)). Polypeptide identifications were accepted if they can be established at greater than 95.0% probability and contain at least two identified peptides. Polypeptide probabilities were assigned by the Protein Prophet algorithm (Nesvizhskii et al., Anal. Chem., 75:4646-58 (2003)). Polypeptides that contained similar peptides and could not be differentiated based on MS/MS analysis alone were grouped to satisfy the principles of parsimony.

### Results

Urine samples were collected from five AL patients, five MM patients, two monoclonal immunoglobulin deposition disease (MIDD) patients, and five control patients (Table 1). The control group consisted of one normal, two patients with monoclonal gammopathy with undeteremined significance (MGUS), and two proteinuric patients with glomerular disease. The diagnosis for the AL was confirmed by renal biopsy using standard immunohistochemistry techniques. One of the MM patients with normal kidney function did not undergo a renal biopsy. The other MM patient had cast nephropathy confirmed on her renal biopsy.

TABLE 1

| | | | | | | Protein- |
|---|---|---|---|---|---|---|
| | | | | | sFLC | uria |
| | Age | Sex | Disease | M-protein | (mg/dl) | (g/d) |
| AL-ex1 | 74 | M | AL | IgGλ + λ | 13.0 | 9.7 |
| AL-ex2 | 51 | F | AL | IgGλ | 8.67 | 1.0 |
| AL-ex3 | 61 | M | AL | λ | 1.19 | 1.7 |
| AL-ex4 | 66 | M | AL | IgGλ | 2.85 | 4.2 |
| AL-ex5 | 53 | M | AL | IgMκ | 4.36 | 8.3 |
| MM-ex1 | 32 | M | MM | IgGκ | 1.41 | 0.03 |
| MM-ex2 | 66 | M | MM | IgAκ | 53.5 | 0.5 |
| MM-ex3 | 65 | M | MM | κ | 105 | 0.4 |
| MM-ex4 | 61 | M | MM | IgGλ | 48.3 | 0.3 |
| MM-ex5 | 41 | F | MM + cast | IgGλ + λ | 758 | 8.9 |
| | | | nephropathy | | | |
| C-ex1 | 57 | F | Membranous | none | n/a | 0.8 |
| | | | glomerulo- | | | |
| | | | nephritis | | | |
| C-ex2 | 54 | F | MGUS | IgGλ + λ | 4.53 | 2.8 |
| C-ex3 | 28 | M | IgA | none | n/a | 1.0 |
| | | | nephropathy | | | |
| C-ex4 | 42 | M | MGUS | λ | 445 | 0.1 |
| C-ex5 | 42 | F | Normal | none | n/a | n/a |

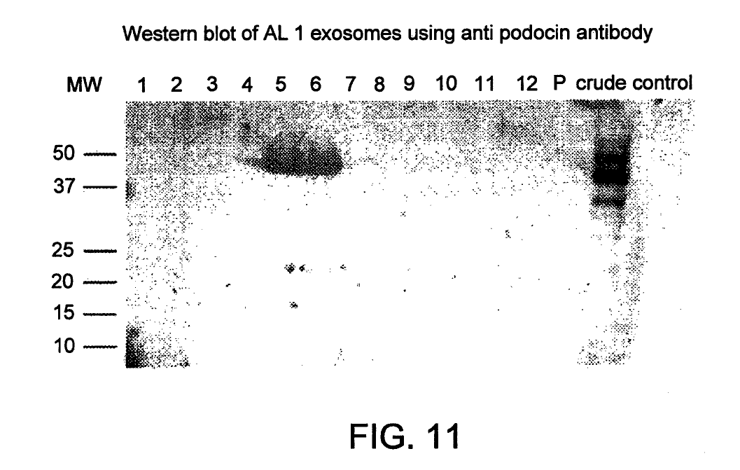

Gradient fractions containing different populations of exosomes were evaluated by Western blotting using free light chain and other antibodies. Fraction 5 and 6 were positive for podocin suggesting a glomerular origin (FIG. 11). Fraction 2 corresponds to collecting ducts with immunoreactivity to aquaporin-2, and fractions 8-10 correspond to distal tubule exosomes with polycystin-1 immunoreactive protein. The polycystin-1 immunoreactivity was confirmed. The SDS-PAGE western blots showed that free lights chains were present in exosomes from all AL, MM, one of the proteinuria patients, and one of the MGUS patients. All samples showed that the majority of the exosome fractions had the expected 25 KDa band corresponding to the light chain monomer although the fractions containing the highest concentration of monoclonal protein varied among the different diseases.

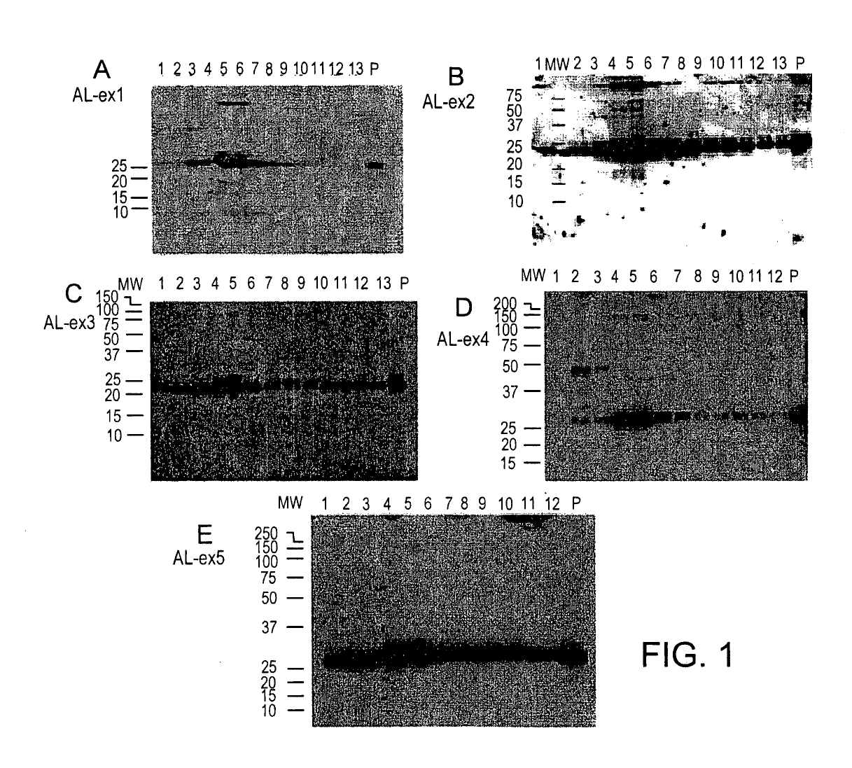

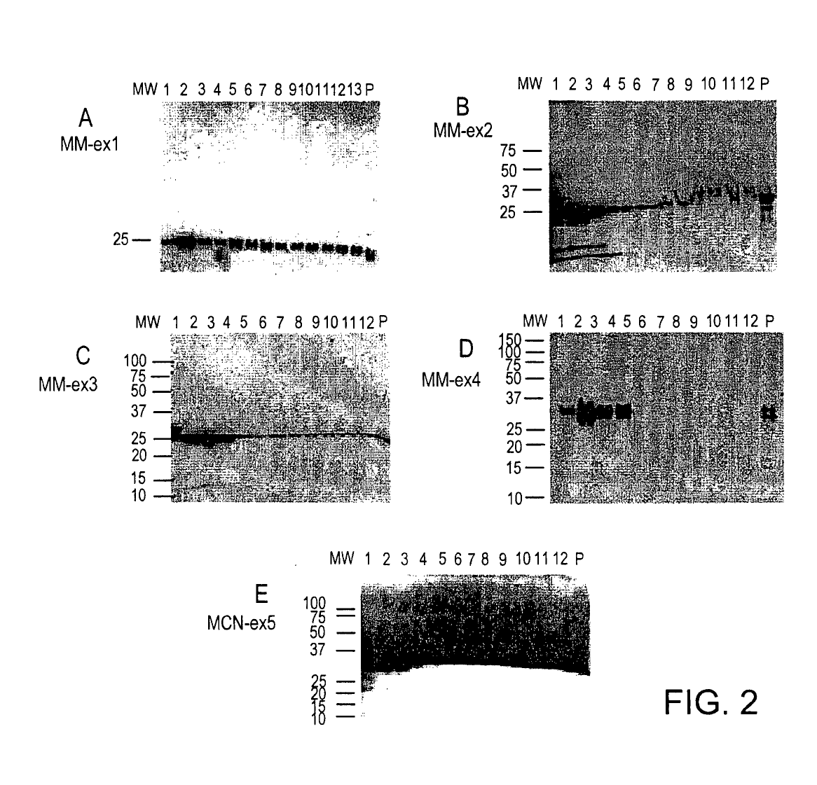

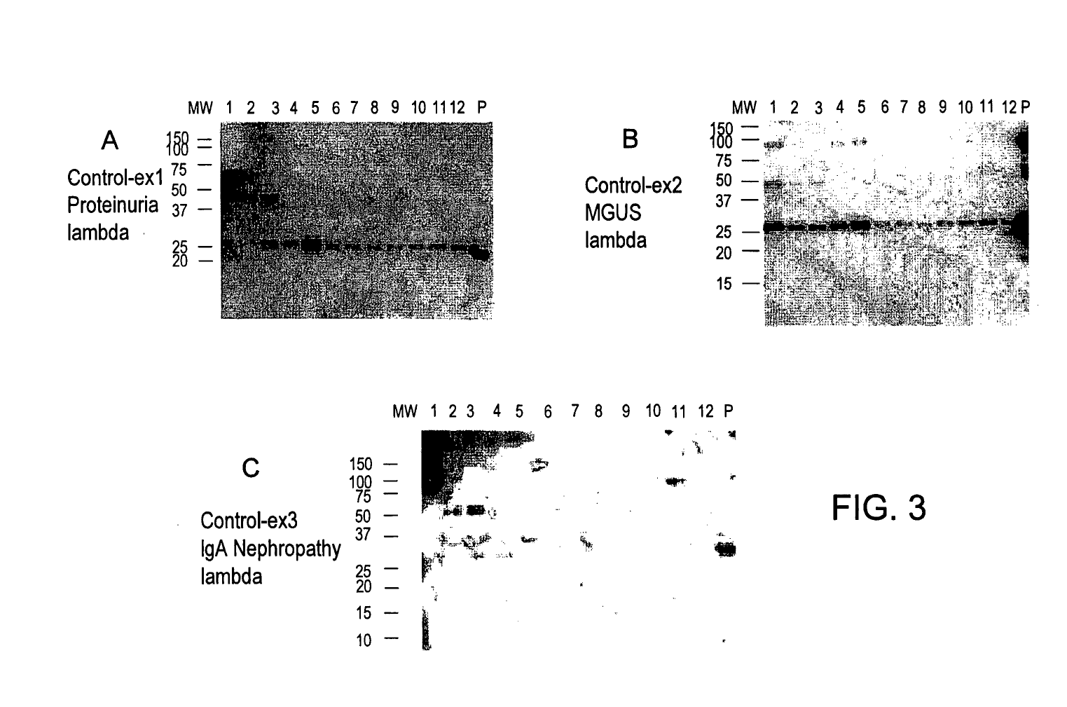

The AL samples exhibited the highest concentration of light chains in fraction 5 and 6 (FIG. 1), while the MM sample had the highest concentration in fraction 2 (FIG. 2). Control patient #1 (proteinuria) had the highest concentration on fraction 5, while control patient #2 (MGUS) had high concentrations in fractions 1 and 5 (FIG. 3). High molecular weight bands were present in the exosomes of AL patients that were immunoreactive for light chains. The maximum molecular weight detected was around 250 KDa corresponding to a decamer (FIG. 1). These high molecular weight bands were not detected in the SDS-PAGE western blots of the MM or controls. Dimers (−50 KDa) were occasionally detected in patients with monoclonal lambda light chains, which are expected since lambda can exist in dimeric state (FIG. 3).

The antibodies used here can detect high molecular weight bands on western blot (Bradwell, Clinical Chemistry, 47:4 673-680 (2001)). Since most of the patients' serum presented with a monoclonal immunoglobulin molecule in addition to the monoclonal light chain, the following was completed to determine if the high molecular weight bands observed were indeed light chains or immunoglobulin molecules. Gradient gels 4-15% were used to separate the high molecular weight bands more accurately with the five AL and five MM samples with the highest concentration of light chain by WB (fraction 5 for AL samples, fraction 2 for MM samples). A western blot was performed using a polyclonal antibody against human IgG. The AL samples from patients with monoclonal IgG (AL patients #1, 2, and 4) and IgM (AL patient #5) exhibited the characteristic bands at 50 kDa (heavy chain only) and 150 kDa (intact immunoglobulin). No IgG molecules were detected in AL patient #3, in agreement with the finding that this patient only has monoclonal lambda light chain. Faint cross reactivity with AL-ex5 IgM heavy chain was detected, possibly due to the denaturing nature of the SDS-PAGE gels. The five MM samples did not show any immunoreactivity with the IgG antibody by western blotting.

These results indicate that the 250 kDa band observed in the AL western blots using free light chain antibody was indeed a decamer of light chains. Moreover, it indicates that the monoclonal IgG in three of the MM samples was not present in their exosomes, offering an additional way to differentiate between AL and MM exosomes.

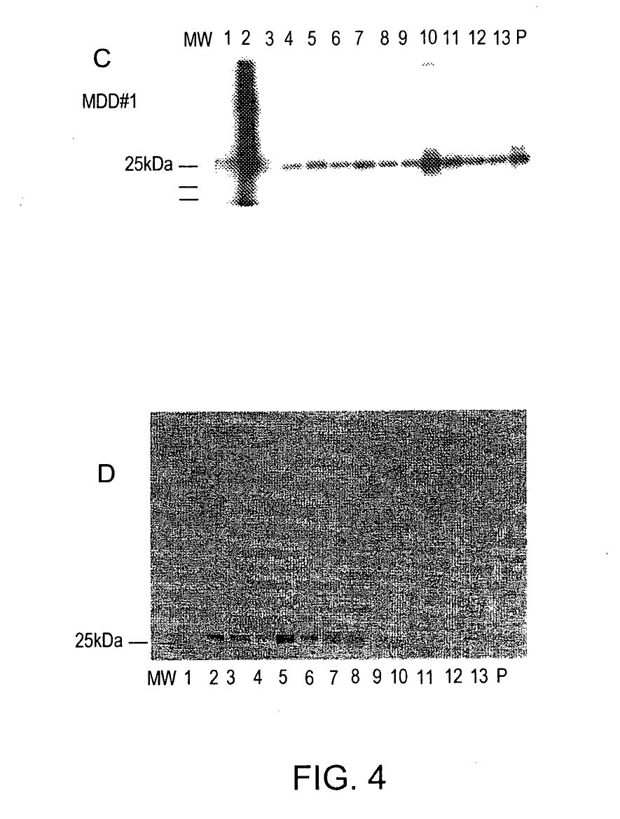

Urine samples were collected from patients with plasma cell dyscrasias. These samples were processed for exosome fractionation using a D2O sucrose gradient as described herein. Gradient fractions containing different populations of exosomes were evaluated using refractometry, silver staining, and Western blotting. Two MIDD samples have been analyzed with the highest concentration of monomeric light chain in fraction 5-6 and fraction 10 (FIG. 4). The sample from MIDD patient #1 exhibited a high molecular weight band that was less intense than the ones observed for AL patients.

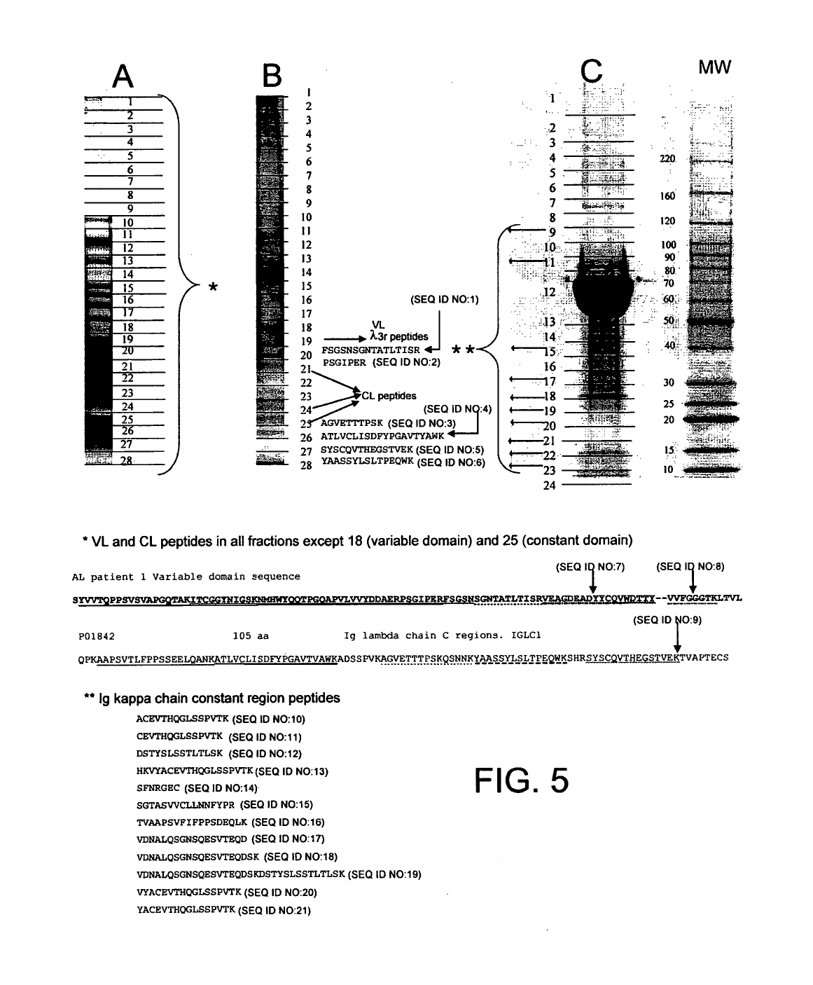

Proteomic analysis of 1D gel slices using mass spectrometry confirmed the results found by Western Blotting. Patient #1 with AL disease possessed light chain polypeptides in every single slice of the gel for both the variable and the constant domain (FIG. 5). MIDD patient #1 presented light chain polypeptides starting at 100 kDa (tetramer).

In general, the polypeptide coverage indicates that the entire light chain is present in the oligomeric species found in exosomes since polypeptides from both variable and constant domains were present in every single slice. Since the process of amyloid formation involves the formation of soluble, oligomeric species (Harperand Lansbury, Annu. Rev. Biochem., 66:385-407 (1997)), this result suggests that the free light chains found in AL exosomes may be part of the amyloid formation process in the kidney.

AL exosomes fractions 8-10 correspond to tubular exosomes with polycystin-1 immunoreactive polypeptide (Hogan et al., J. Am. Soc. Nephrol., 20(2):278-88 (2009) and Sikkink and Ramirez-Alvarado, Amyloid, 15:29-39 (2008)). The fractions that present high molecular weight oligomeric light chain species had podocin present as shown by western blotting (FIG. 11). This indicates that these exosomes are of glomerular origin, the common site of amyloid deposition. None of the other exosomal populations from the different diseases presented podocin immunoreactivity.

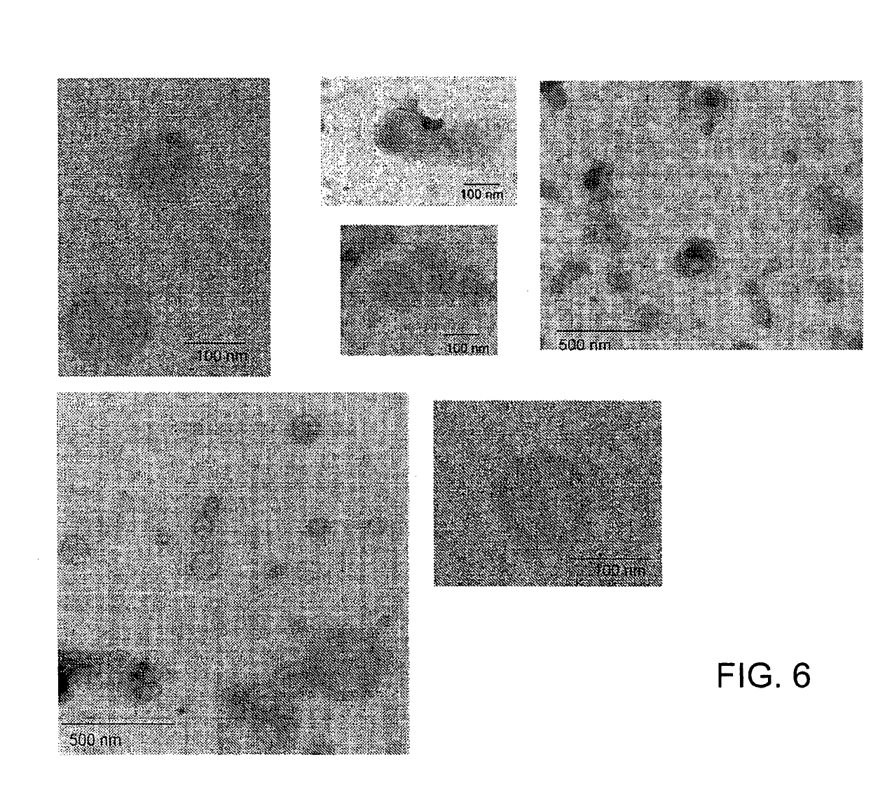

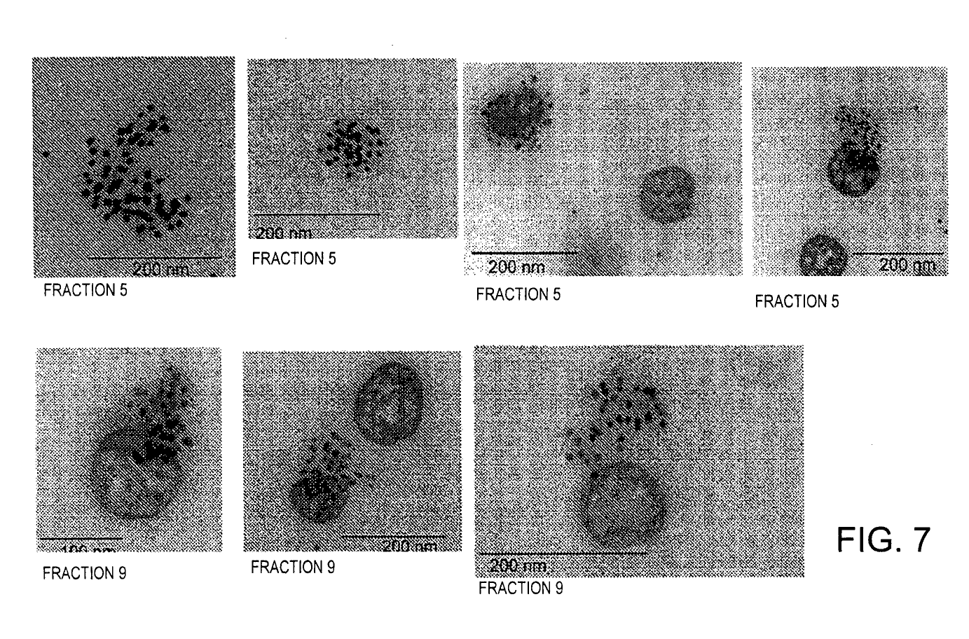

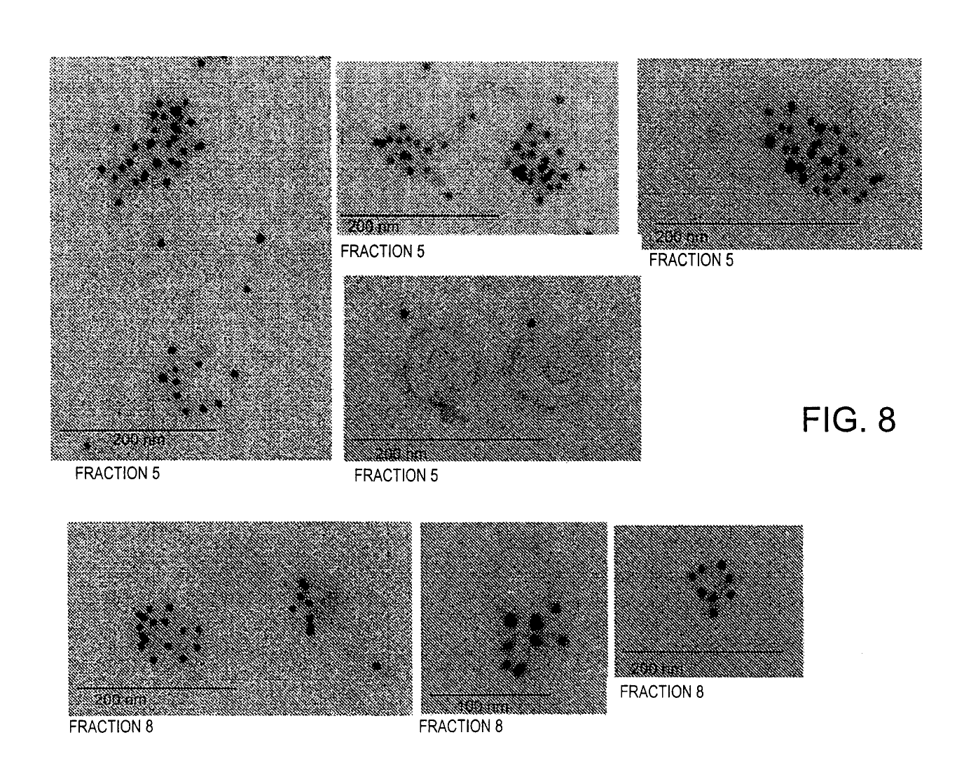

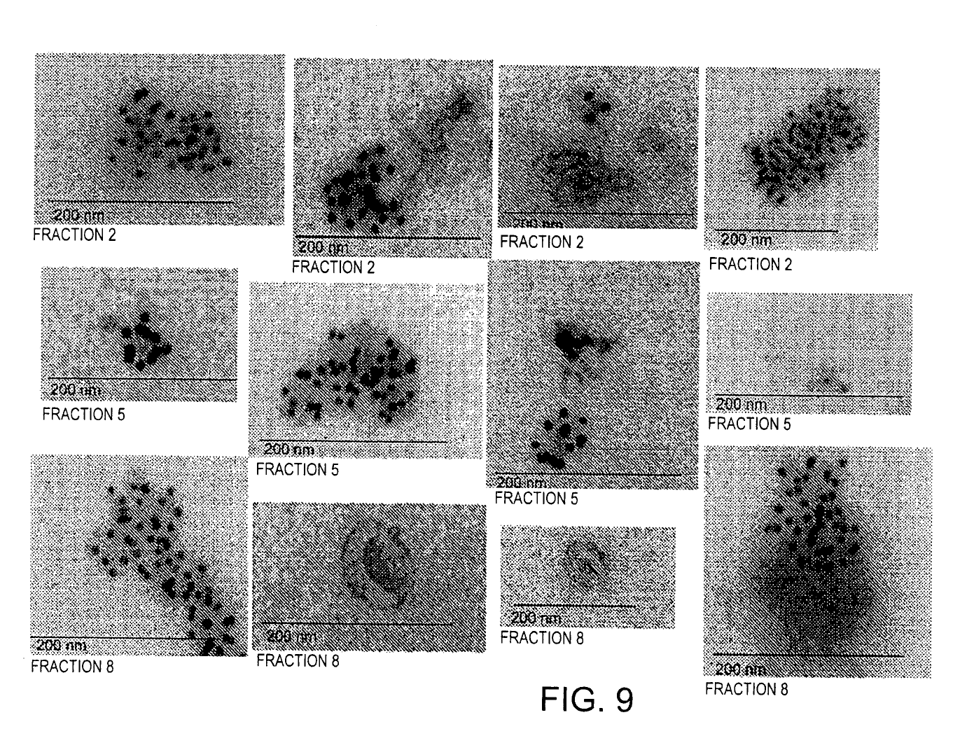

Electron microscopy images of normal exosomes revealed that the morphology of these exosomes was similar to the previously reported ‘deflated football’ with concave and convex surfaces (FIG. 6). Immuno-gold electron microscopy analysis of disease-related exosomes revealed a similar morphology. The diameter varied depending on the fraction of exosomes studied in each disease sample, ranging from 20-100 nm. All exosomes analyzed presented light chains on the surface. AL exosomes exhibited a high concentration of light chain on the membrane of exosomes to the extent that it is difficult to find the membrane limits. Some multivesicular body (MVB) exosomes that presented a distinct enhanced negative staining with multiple vesicles in the interior of the exosome were identified. The majority of MVB exosomes did not present free light chain labeling. In the cases where there was free light chain labeling, it accumulated in a certain region of the exosome surface (FIG. 7). MIDD exosomes present less intense free light chain labeling on the surface (FIG. 8). MVB exosomes were observed with this sample when using fraction 8 staining with polycystin 1 antibody. MM exosomes from fraction 2, 5, and 8 were imaged. These MM exosomes presented similar features of AL and MIDD exosomes, although no MVB exosomes were found in the MM sample (FIG. 9).

To determine the location of free light chains in exosomes (membrane and/or cytosolic), membrane contents were separated from soluble contents, and western blotting using free light chain antibodies was performed. AL exosomes presented free light chains both in the membrane and the soluble exosomes fractions. The normal sample studied exhibited free light chains only in the membrane fraction after a very long exposure compared to the initial gradient analysis. In the case of the AL exosomes, there was an apparent enrichment of light chains within the membrane fraction compared to the other samples analyzed. It is possible that AL exosomes have a predominantly membrane bound light chain population that is further enriched by the separation of the cytosolic components.

A sample from MIDD patient #1 exhibited no soluble protein, while a sample from MIDD patient #2 exhibited about 50% of the protein as being located in the surface of the exosome. The remaining was found in the cytoplasmic portion of the exosome.

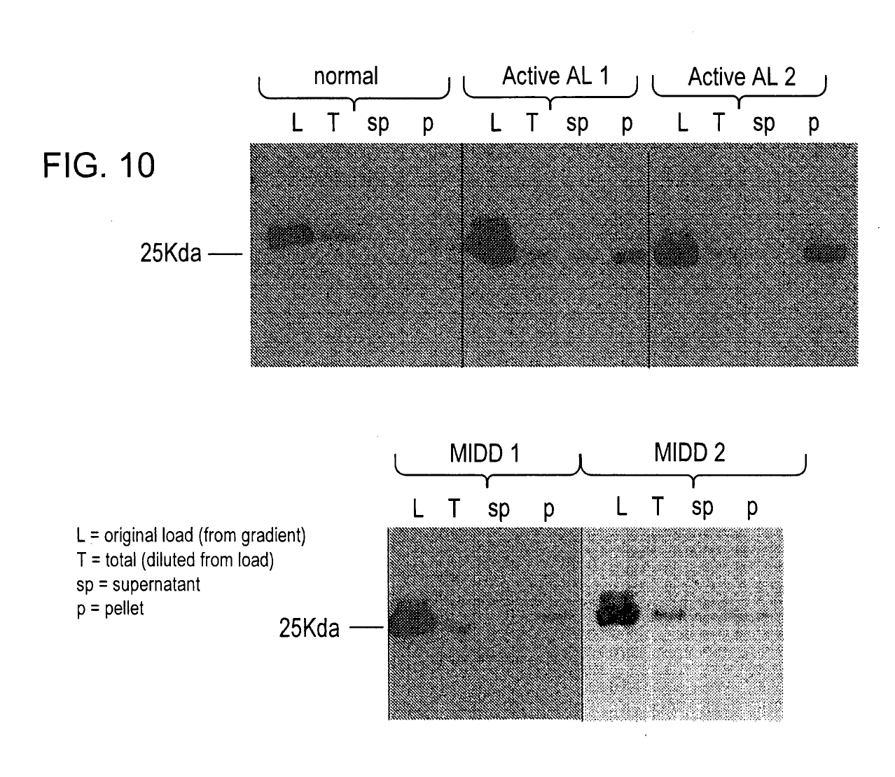

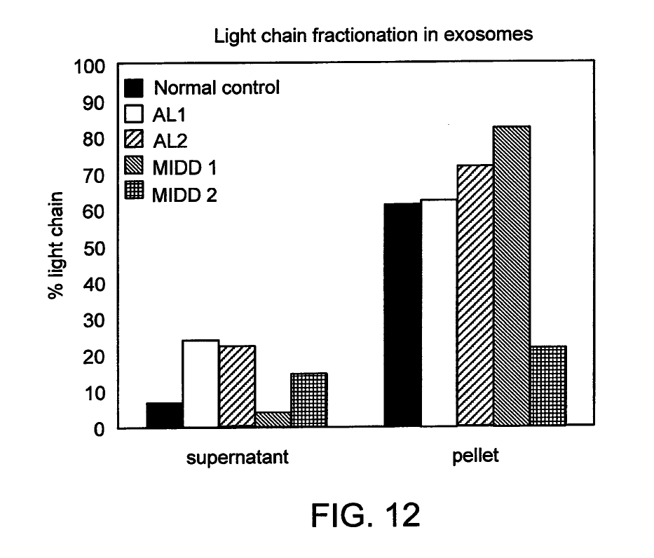

Densitometry analysis of the bands on FIG. 10 revealed that the majority of the free light chains in exosomes was attached to the membrane. AL exosomes presented more light chains in the cytoplasmic fraction than the normal or light-chain deposition disease samples (FIG. 12).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing a mammal for active immunoglobulin light chain amyloidosis, wherein said method comprises determining whether or not a urine sample from said mammal contains exosomes having immunoglobulin light chain polypeptides associated as complexes with a molecular weight greater than 150 kDa, wherein the presence of said exosomes indicates that said mammal has active immunoglobulin light chain amyloidosis, and wherein the absence of said exosomes indicates that said mammal does not have active immunoglobulin light chain amyloidosis.

2. The method of claim 1, wherein said mammal is a human.

3. The method of claim 1, wherein said molecular weight is greater than 200 kDa.

4. The method of claim 1, wherein said exosomes contain a podocin polypeptide.

5. The method of claim 1, wherein said method comprises diagnosing said mammal as having active immunoglobulin light chain amyloidosis if said exosomes are present and diagnosing said mammal as not having active immunoglobulin light chain amyloidosis if said exosomes are absent.

6. The method of claim 1, wherein said determining step comprises using an antibody to detect the presence or absence of said exosomes having immunoglobulin light chain polypeptides associated as complexes with a molecular weight greater than 150 kDa.

7. The method of claim 1, wherein said determining step comprises using an anti-immunoglobulin light chain antibody to detect the presence or absence of said exosomes.

8. The method of claim 1, wherein said determining step comprises performing a Western blot.
